(19) United States
(12) Patent Application Publication
(10) Pub. No.: US 2006/0051875 A1
(43) Pub. Date: Mar. 9, 2006

(54) Method for detecting a plurality of different species

(75) Inventors: Mary A. Reppy, Wilmington, DE (US); Charles F. Saller, Escondido, CA (US)

Correspondence Address: CONNOLLY BOVE LODGE & HUTZ LLP, SUITE 800, 1990 M STREET NW, WASHINGTON, DC, 20036-3425, US

(73) Assignee: Analytical Biological Services, Inc., Wilmington, DE (US)

(21) Appl. No.: 11/198,164

(22) Filed: Aug. 8, 2005

Related U.S. Application Data
(60) Provisional application No. 60/599,030, filed on Aug. 6, 2004.

(51) Int. Cl. G01N 33/48

(52) U.S. Cl. 436/164

(57) ABSTRACT

Two-dimensional and/or three-dimensional polymeric or extended solid arrays, such as arrays of a polydiacetylene backbone, are used to screen a plurality of samples containing different species by monitoring the change in the fluorescence or phosphorescence of the array upon exposure to the sample and comparing it to a known change in fluorescence or phosphorescence, respectively.

### TECHNICAL FIELD

The present disclosure relates to a method for detecting a plurality of different species and especially in assays useful in the pharmaceutical industry. More particularly, the present disclosure involves a method using two and/or three-dimensional polymeric or extended solid arrays and preferably using polydiacetylene arrays. The arrays are exposed to the sample to be tested and the fluorescence or phosphorescence of the arrays measured and compared to arrays exposed to a standard. The arrays respond to many different analytes giving heterodetection of different species, rather than detection of a single analyte.

### BACKGROUND OF THE INVENTION

Polydiacetylenes are conjugated polymers with backbones of alternating double and triple bonds formed from the 1,4-addition polymerization of 1,3-diacetylenes. Polydiacetylenes generally absorb well in the visible region of the spectrum, and hence are highly colored, ranging from blue to yellow. There has been intense interest in the non-linear optic properties of polydiacetylenes and extensive study has been made of both the solvo-chromic properties of solubilized polydiacetylenes and the thermochromic properties of polydiacetylene films and single crystals. It is well known that to form polydiacetylene, the diacetylene monomers must be in an ordered packing to allow the polymerization to occur. It seems to be generally accepted, though the inventors are not bound herein, that the packing of the side chains can affect the conjugation length of the backbone, and hence the chromic and emissive properties.

Diacetylene monomers have been used to form various ordered systems, including crystals, liquid crystals, liposomes and films that were then polymerized to form the polymer. Liposomes have been made from monomers with two diacetylene chains and polar head groups (such as phosphotidylcholines, and its analogues) and from monomers with single diacetylene chains. The liposomes can be polymerized with UV light or γ-radiation. Monomer films have been formed by Langmuir Blodgett methods or cast from solvents and then also polymerized with UV light or γ-radiation. The choice of monomer structure, conditions of liposome or film formation, and polymerization conditions all affect the conjugation length of the polydiacetylene backbone, and hence the color of the system. Upon heating, these polymerized systems can undergo a change in the effective conjugation length, from the longer length forms (blue and purple) to the shorter length forms (red and yellow). This change has been attributed to the side-chains moving and repacking upon being heated. Soluble polydiacetylenes show solvo-chromic behavior and polydiacetylene films often change color upon exposure to solvent vapors. Polydiacetylene films and liposomes formed from diacetylene surfactants also often change color with change in pH. In the case of the packed polymer arrays that form the films and liposomes, it is generally accepted that changes in the environment that affect the organization and packing of the side chains coming off the conjugated backbone can affect the conjugation length and hence the chromic and electronic properties of the polymer.

The phenomenon of fluorescence is distinct from the absorbance properties that give systems their color. The colors we see are related to the wavelengths of light that the species is absorbing. For example if the species absorbs light primarily at 650 nm, we will see it as blue, while if it absorbs primarily at 550 nm, we will see it as red. Color arises from absorbance of light in the visible range. Most colored species are not fluorescent. In order to be fluorescent, the system must absorb one wavelength of light and then emit another. Upon absorbing the light, the system is excited to a higher energy state. It can then return to the ground state by a variety of mechanisms, most of which do not lead to fluorescence. These alternative, non-radiative, mechanisms for returning to the ground state lead to many strongly absorbing species to be non-fluorescent, and makes the prediction of which species will be fluorescent a difficult task and therefore not apparent to those skilled in the art.

For instance, while some organic systems with extended conjugation exhibit fluorescence, many more do not.

Polydiacetylenes can show fluorescence. However, their ability to fluoresce is dependent on the structural form and organization of the polymers, particularly the conjugation length and aggregation state, whether in solution, a film, or formed into liposomes or other three-dimensional structures.

It is known that polydiacetylene films have an intrinsic fluorescence when produced in the red or yellow form, and are not fluorescent (by conventional measurements) when the film is made in the blue form (Yasuda A. et al, Chem. Phys. Lett., 1993, 209(3), 281-286). This fluorescent property of the films has been used for microscopic imagining of film domains and defects.

Ribi et al have suggested two sensors using polydiacetylene film fluorescence. The first sensor (Saul et al, U.S. Pat. No. 5,415,999 and U.S. Pat. No. 5,618,735) uses a red, fluorescent, polydiacetylene film layered with a fluorescence modulation reagent non-covalently associated with the film that modulates the measured emission of the film, e.g. by absorbing the emitted light, in the presence of an analyte. The fluorescent state of the film does not change during the assay; rather the emission is obscured or revealed by the action of the fluorescence modulation agent. The second suggested sensor (Ribi, U.S. Pat. No. 5,622,872) uses a film of specific composition for detection of an analyte by change in the fluorescence of a film of this composition. The films in the detection method claims comprise a polymerized film, polymerized from diacetylene monomers of the defined formulation (A)ₐ(D)ₐCₓ(C≡C)₂C_{y}LB wherein A is a functional group used to link the film to an underlying substrate, a is 0 or 1, C is carbon, x and y are 1 or greater and (x+y) is in the range of 4-32, D and L are bond or linking groups and B is a specific binding member which binds to a specific analyte, one terminus of each monomer is proximal to the underlying substrate and the other terminus comprising B (i.e. the film is a mono-layer with every polydiacetylene side-chain either terminating in proximity to the underlying substrate, or in a binding member). Neither Ribi nor others, to knowledge of the present inventors, have suggested detection of multiple compounds in a non-specific fashion using three-dimensional or two-dimensional arrays of polydiacetylenes and measuring the emission.

More recently the present inventors have discovered that the change in polydiacetylene arrays from a non-fluorescent to a fluorescent state can be used for selective detection of an analyte by measuring the emission of an array incorporating a ligand, receptor or substrate specific for the analyte. Furthermore the extent of this change can be magnified by incorporation of suitable fluorophores. These discoveries are described in our previous patent application (Reppy M. A., Sporn S. A., Saller C. F., “Method for detecting an Analyte by Fluorescence”, PCT International Patent WO/00171317, and U.S. patent application Ser. No. 09/811, 538), disclosures of which are incorporated herein by reference.

The present inventors have also discovered that polydiacetylene arrays can be used for evaluating compounds log P, oral absorption and cellular permeability. The change in fluorescence or phosphorescence of the array is measured or detected and compared to the change in fluorescence or phosphorescence, respectively, of identical arrays exposed to standard or reference compounds in solution. This comparison can be used to evaluate the organic/water partition coefficient and lipophilicity or the likely oral absorption of the compound or their transcellular permeability. The method can also be used to assess the binding of compounds to proteins or other macromolecules. These discoveries are described in our previous patent application (Reppy M. A., Saller C. F., “Method for Evaluating Drug Candidates”, U.S. patent application Ser. No. 10/420,807, filed Apr. 23, 2003 and PCT International Patent).

### SUMMARY OF INVENTION

The present disclosure provides materials and a method for the detection of chemicals in a non-specific or hetero fashion by measuring the effect of the chemicals on the fluorescence or phosphorescence of two-dimensional or three-dimensional polymeric or extended solid arrays. The term “hetero-detection” refers to detecting multiple species rather than being selective for one specific species. More particularly this disclosure provides for the detection of compounds in screening assays used for evaluation of possible drug candidates.

The present disclosure provides a method for screening a plurality of samples containing different species, which comprises exposing a three-dimensional array of a polydiacetylene backbone or a two-dimensional array of a polydiacetylene backbone, or both, to the samples to be evaluated; wherein the array is capable of hetero-detection:
detecting the change in fluorescence or phosphorescence of the array, and
comparing the change to a previously determined change in fluorescence or phosphorescence of the array to determine whether the species are present in the samples. In a further refinement, comparison with calibration curves allows determination of the concentration of the species.

### BEST AND VARIOUS MODES

Two-dimensional and three-dimensional arrays employed according to certain embodiments of this disclosure comprise a polydiacetylene backbone. The arrays can be prepared by polymerization of precursor diacetylene arrays. The diacetylene precursor two and three-dimensional arrays may also contain species that are not diacetylenes.

The polydiacetylene backbones employed according to these embodiments are known and need not be described herein in any detail and can range from being oligiomeric (from the reaction of three or more monomers) to polymeric. For example see U.S. Pat. No. 6,001,556 to Charych et al, disclosure of which is incorporated herein by reference.

In this embodiment the polydiacetylene is formed from polymerizing a three-dimensional or two-dimensional array of diacetylenes. The array may also contain non-diacetylene species such as natural and unnatural phospholipids, cholesterol, lipids, proteins and other species including charged and hydrogen-bonding species. The array may also contain other non-diacetylene species and multiple diacetylene species.

Also, side chains with ordering head groups are typically bound to the polydiacetylene backbone. The head groups are typically polar.

The arrays according to certain embodiments can be formed by polymerizing arrays of diacetylene monomers. The typical monomers are single or multi-tailed diacetylene surfactants with polar head groups. More typically used are single or bis-tailed diacetylene surfactants with polar head groups. There may be polar head groups on both ends of a single chain diacetylene species. Embodiments according to this disclosure do not dependant on use of any specific diacetylene surfactant, tail structure, or polar head group, but can be used with any diacetylene monomer that can be polymerized to give polydiacetylene in its non-fluorescent form or polydiacetylene in a fluorescent form that can converted to another fluorescent form with a different magnitude of emission.

Materials typically used as head groups include, but are not limited to: carboxylic acids, carboxylate salts, amides, ethanol amide, amines, ammoniums, imines, imides, alcohols, carbamates, carbonates, thio-carbamates, hydrazides, hydrazones, phosphates, phosphonates, phosphoniums, thiols, sulfates, sulfonates, sulfonic acids, sulfonic amines, sulfonamides, amino acids (including glutamate and glutamine), peptides, nitro-functionalized moieties, carbohydrates, choline, ethylene glycol, oligiomeric ethylene glycol, poly(ethylene glycol), propylene glycol, oligiomeric propylene glycol, and poly(propylene glycol), and combinations thereof.

When the arrays are to be secured or anchored to a support surface, the tails and/or head groups of the lipids can be selected to provide this function.

The two-dimensional and three-dimensional arrays can be produced in any number of forms. Liposomes are one of the suitable three-dimensional array forms that can be produced. The liposomes can be formed in a number of different sizes and types. For instance, it is possible to form the liposomes as simple bi-layer structures. Liposomes can also be multi-layered with an onion type structure. Their size can also be varied. Tubules, tube, ribbons and fibers are other suitable three-dimensional forms. A suitable two-dimensional array form that can be produced is a film. The film can be mono-layered, bi-layered, or multi-layered.

Numerous other shapes can also be produced. Lamellae (Rhodes et al, Langmuir, 1994, 10, 267-275), hollow tubules and braids (Frankel et al, J. Am. Chem. Soc., 1994, 116, 10057-10069.), ribbons, crystals, lyotropic and thermotropic liquid crystalline phases, gels and amorphous structures are among the other shapes that can be formed. When these assemblies are immobilized they can collectively form even larger constructs.

Polydiacetylene can also be formed as fluorescent and non-fluoresent gels with a net-work structure of aggregated fibers. Polydiacetylenes can be used in the formation of composite materials, including layering with inorganic clays. Film arrays of diacetylenes or polydiacetylenes may be used in the free standing form, or supported on glass, ceramic, polymer, paper, metal, or other surfaces. The supports may be porous, including, but not limited to, nano and micro porous membranes. Diacetylene coatings may also be cast onto glass, ceramic polymer, paper, metal or other surfaces and photopolymerized to give the polydiacetylene arrays described above.

Diacetylene and polydiacetylene liposomes or other colloidal structures may be attached to, supported on, or absorbed in, solids, including, but not limited to: polymers such as polystyrene, polycarbonate, polyethylene, polypropylene, cellulose, cellulose esters, nylon and polyfluorocarbons such as Teflon® (polymers of tetrafluoroethylene), perfluorinated ethylene-propylene copolymers, copolymers of tetrafluoroethylene and perfluoroalkoxy, copolymers of tetrafluoroethylene and ethylene, polymers of chlorotrifluoroethylene, and copolymers of chlorotrifluoroethylene and ethylene; silicon chips; beads; filters and membranes; glass; gold; silica; sephadex; sepharose; porous or swelling solids such as polyacrylates and polyacetonitrile; and sol-gels. In the case of diacetylene liposomes and film arrays, they can be polymerized after incorporation with or attachment to the solid support.

An embodiment of solid supported polydiacetylenes is as an array on nano-porous membranes. We have discovered that diacetylene liposomes and other colloidal structures can be forced in and onto membranes including 100, 200 and 400 nm membranes and photopolymerized to create non-fluorescent polydiacetylene. These coated membranes are stable at room temperature, in air, and exposed to light, for at least 12 months. The polydiacetylene array coating exhibits some resistance to abrasion. The polydiacetylene arrays can be converted from the non-fluorescent to the fluorescent form or from one fluorescent form to another fluorescent form with a different magnitude of emission or the fluorescent to the non-fluorescent form in response to environmental changes including exposure to a solution containing a test compound. In other words, the fluorescence of the arrays is either reduced or enhanced by exposure to the test sample.

Nanoporous membranes are available in many materials, including: alumina, polyfluorocarbons such as Teflon® (polymers of tetrafluoroethylene), perfluorinated ethylene-propylene copolymers, copolymers of tetrafluoroethylene and perfluoroalkoxy, copolymers of tetrafluoroethylene and ethylene, polymers of chlorotrifluoroethylene, and copolymers of chlorotrifluoroethylene and ethylene; nylon, polycarbonate, cellulose, cellulose esters, polyvinylene difluoride (PVDF), and glass and also in a variety of pore sizes. Use of any of these membrane types with pore sizes typically up to about 600 nm are envisioned for preparing solid supported polydiacetylenes. Microtiter plates are available and can be made with nanoporous membranes for the well bottoms that can be precoated with the diacetylene or polydiacetylene arrays or coated with the diacetylenes arrays in situ and polymerized.

By way of example, the diacetylene two-dimensional and three-dimensional structures are photopolymerized with UV light, or γ-radiation, to give organized polydiacetylenes with the longer conjugation lengths characterized by absorption maximum in the range of 500-800 nm, more typically in the range 600-750 nm, and a blue to purple color. The photopolymerization results in creating mainly the non-fluorescing form and therefore exhibiting low overall fluorescence relative to the background. The term “non-fluorescent form” as used herein also refers to these polymers which have low overall fluorescence exhibiting a fluorescent signal above 500 nm that is only about 1-3 times that of the background and less than that of the corresponding fluorescent form. Typically the “non-fluorescent form” exhibits a fluorescent emission above 500 nm that is at least about 10% lower and more typically at least about 50% lower than that of the corresponding fluorescent form. Some diacetylene two-dimensional and three-dimensional arrays give polydiacetylene in the fluorescent forms upon photopolymerization; these may still be used in assays if interaction with a test compound converts the arrays to a fluorescing form having a different measurable emission that is either lower or higher than the original emission. The arrays may also be heated or exposed to chemicals to convert the polydiacetylene to the fluorescent form.

In the application described here, the exposure of polydiacetylene arrays to compounds in solution can cause a drop or a rise in fluorescence. The arrays are designed to respond to a large range of diverse compounds, rather than being specifically targeted to one compound. In previous work, the polydiacetylene arrays were formulated and designed to respond to different species by a change in fluorescence emission in a differential fashion, either targeting specific entities or distinguishing between compounds with specific characteristics. Here, the arrays are designed and formulated to change their fluorescence emission in response to the presence of many different compounds, presented individually or in mixtures, in an indiscriminate fashion. This is important for the utility of the arrays for detection of compounds in screening assays wherein it may be desired to screen hundreds to thousands of compounds of many different types. According to the present disclosure, the method is intended typically to be used in screens for at least five different compounds, more typically at least about twenty different compounds, even more typically at least about one hundred and even about one thousand or more different compounds.

The polydiacetylene fluorescence of the fluorescent form may be excited by light with wavelengths between 300 and 600 nm, and consists of a broad fluorescence above 500 nm with one or two maxima though the disclosure is not bound by these specifics.

It seems likely, though the inventors are not bound herein, that rise in the fluorescence emission can be a result of either a rise in the population of shorter conjugation length polydiacetylene backbones, or a decrease in the longer conjugation length polydiacetylene backbones, or a change in the aggregation of the backbones, or combination of these and other factors. The relative change in fluorescence can be an order of magnitude, or more, greater than the relative changes measured in the UV/VIS absorption spectrum upon this transformation. This means that fluorescence can provide a more sensitive measure of change in the liposomes than the direct chromic response. This increase in sensitivity makes the novel fluorescence detection method of use in many areas where colorimetric detection would simply not be sufficient. These include drug discovery assays. For films, the fluorescent/non-fluorescent properties of the polydiacetylenes can be used as a detection method and would also provide increased sensitivity compared to calorimetric detection in immobilized sensing systems. Fluorescence detection also allows sensing platforms to use opaque supports, whereas colorimetric detection requires transparent (e.g. quartz glass or UV/VIS-transparent plastic) supports.

In a further embodiment, the arrays may incorporate other fluorescent species. These other fluorophores may be organic, biological, inorganic or polymer compounds, complexes or particles. They may be attached to the surface of the arrays or incorporated in the interior. Many of the fluorophores are lipophilic and are expected to incorporate into the alkyl region of the liposome, while others are polar or charged and are expected to end up in the head group region/aqueous interface, or in the water solution. The fluorophores can enhance the magnitude of the change in the fluorescence of the polydiacetylene arrays as they change from the non-fluorescing to the fluorescing form. The fluorescence of these fluorophores can also be monitored during this conversion, either as an internal standard if the fluorescence of the fluorophore is not affected by changes in the polydiacetylene, or as an additional measure of the conversion when the fluorescence of the fluorophore does change. In addition certain fluorophores can undergo excited state energy transfer processes that change the overall fluorescence of the array, and increase the quantum yield.

For instance, the added fluorophores may optically and/or electronically interact with the polydiacetylene polymer. There are several ways that the fluorophores and the array can optically/electronically interact, including but not limited to the following:
(1) By the fluorophore absorbing the fluorescence of the array or by the array absorbing the fluorescence of the fluorophore.
(2) By the fluorophore absorbing the excitation light, becoming excited and then transferring energy from its excited state to the polydiacetylene array causing it to fluoresce. This process is known as Resonance Energy Transfer (RET) and also as Fluorescence Resonance Energy Transfer (FRET). This RET process allows the polydiacetylene fluorescence to have the time decay properties of the fluorophore.
(3) By the polydiacetylene array, in its fluorescent form, absorbing the excitation light and then transferring the energy from its excited state to the fluorophore leading to the fluorophore fluorescing. This RET process can lead to an increase in the effective Stokes shift of the system and also increase the overall quantum yield.
(4) By the excited state of the fluorophore transferring an electron to the array or by the excited state of the array transferring an electron to the fluorophore. This process is known as Photoinduced Electron Transfer (PET).
(5) By the array absorbing the excitation light needed for fluorophore fluorescence or the fluorophore absorbing the excitation light needed for array fluorescence.
(6) By the fluorophore quenching the fluorescence of the array.
(7) By the array quenching the fluorescence of the fluorophore.

Addition of fluorophores to the polydiacetylene array can make it possible to increase the extent of the change in fluorescence of the array during an assay, thus increasing assay sensitivity; and also to monitor the fluorescence of the fluorophore during the assay as a second measure of change in the polydiacetylene array caused by the analyte.

The fluorescence of the arrays can be read with any equipment known in the art for fluorescent measurements, including, but not limited to, fluorometers with cuvette and fiber optic attachments, plate readers, hand held readers, fluorescence microscopes, CDC cameras, and by eye. This sensing method may be readily used in the multi-well plate formats of high-throughput screening.

Presented below are examples of applications for drug discovery and development as examples of uses of the polydiacetylene 3-dimensional or 2-dimesional arrays for non-specific or hetero-detection of compounds in assays.

Many of these example applications are concerned with the determination of ADME (i.e., absorption, distribution, metabolism, excretion) properties of compound collections that are screened to obtain potential drug leads/candidates. Many compounds that are effective for a particular therapeutic target fail because of problems with their ADME properties. Poor solubility of drug candidates in water, inability to cross cell membranes and high binding affinity to serum proteins are three defects that cause otherwise promising candidates to fail as therapeutic agents. Therefore, pharmaceutical companies are increasingly looking for ways to examine these properties early on in the drug discovery process. Several different assays have been developed for screening compounds for drug-like properties that require a method for detecting the presence of the compound in a solution after it has passed through a filter or membrane.

The application examples presented below show the use of the 3-dimensional and 2-dimensional polydiacetylene array formulations that non-specifically detect compounds in assays for measuring compound permeability across filter supported artificial membranes (as a measure of bioavailability), compound solubility, and compound binding to human serum albumin. Assays to measure these properties are in place throughout the pharmaceutical industry, however, current methods have significant problems that limit their utility. In these assays, known compounds are added under various test conditions and then the concentration of the compound that crosses a filter membrane, or an artificial biomimetic membrane, or remains unbound to proteins is measured. The identity of the compounds are known, only the measurement of their concentrations are needed, which could be either measurement of compound concentration in a concentration dependent manner or simply the detection of specific concentration threshold. For many compounds this is currently accomplished by means of UV absorbance measurements. However, this does not work with a very large number of compounds (˜20%), necessitating the use of time-consuming and labor intensive measures such as LC-MS. Furthermore, even when UV absorbance measurements are possible, they require full scans of UV absorbance to determine the optimal wavelength for measuring the compound concentration, making this step considerably slower than with fluorescence measurements such as described in this application. The method that is described herein addresses this problem and greatly facilitates HTS compound screening.

The following non-limiting examples are presented to further facilitate an understanding of the present disclosure.

### EXAMPLE 1

One example of a permeability assay using the polydiacetylene arrays to detect compounds would be as follows:
1. A known concentration of a specific compound is added to the upper chamber of a two-stage 96- or 384-well microtiter plate. The bottom of the upper stage consists of a permeable artificial membrane, through which compounds may enter the low stage containing acceptor wells containing 3-dimensional or 2-dimensional polydiacetylene arrays.
2. After an appropriate incubation time, the two plate stages are separated and the amount of compound in the lower stage is quantified by measuring the fluorescence emission of the arrays in a standard microtiter plate reader and comparing the emissions to the emission of the arrays exposed to a reference solution, or to the emission of the arrays measured before the start of the assay. A calibration curve may be used to determine the concentration of the compound.

### EXAMPLE 2

An example of use of the polydiacetylene arrays in a human serum albumin (HSA) binding assay is as follows:
1. A known concentration of a specific compound is added to the upper chamber of a two-stage 96- or 384-well microtiter plate containing human serum albumin. The bottom of the upper stage consists of a micro-porous membrane, through which compounds may enter the lower stage plate acceptor wells containing 3-dimensional or 2-dimensional polydiacetylene arrays.
2. After an appropriate incubation time, the microtiter plates are briefly centrifuged to separate the compound that is bound to the HSA, which is retained in the upper chamber, from free compound, which is collected in the lower chamber containing the 3-dimensional or 2-dimensional polydiacetylene arrays. In a variation of this example, the arrays may be added after the centrifugation step. The two plate stages are separated and the amount of compound in the lower stage is quantified by measuring the emission of the arrays in a standard microtiter plate reader and comparing the emissions to the emission of the arrays exposed to a reference solution, or to the emission of the arrays measured before the start of the assay. A calibration curve may be used to determine the concentration of the compound.

### EXAMPLE 3

An example of use of the polydiacetylene arrays in a compound solubility assay is as follows:
1. A known amount of compound is added to a volume of buffer. The mixture is shaken for a set period of time and then filtered through a filter that catches undissolved compound. The receiving chamber for the filtrate contains 3-dimensional or 2-dimensional polydiacetylene arrays; alternatively the arrays are added after the filtration step. This assay may be run in a 96-well filter plate with the filtered compound collected into another 96-well plate.
2. The amount of compound that is dissolved is quantified by measuring the emission of the arrays and comparing the emissions to the emission of the arrays exposed to a reference solution, or to the emission of the arrays measured before the start of the assay. A calibration curve may be used to determine the concentration of the compound.

### EXAMPLE 4

An example of polydiacetylene liposomes responding to multiple drug and drug-like compounds in a relatively indiscriminate fashion is given here. The test compound set consisted of: amiodarone (AMIO); clofazimine (CLOF); pimozide (PIM); terfenadine (TERF); triflupromazine (TRIF); chlorpromazine (CPZ); beta-estradiol (ESTR); flavone (FLAV); ketoprofen (KETO); furosemide (FURO); hydrocortisone (HCOR); procainamide (PROC); cimetidine (CIM); hydrochlorothiazide (HCHL); and tetracycline (TCYC).

Liposomes were prepared from 10,12-pentacosadiynoic acid (PDA), from GFS Chemicals, with 5% BODIPY™ TR-Cadaverine (1), from Molecular Probes, incorporated in 2.0 mM HEPES buffer at pH 7.4, and polymerized with 0.4 J/cm² of UV light around 254 nm. The wells of a black polystyrene 384 well plate were charged with 4 μL of liposome solution, 32 μL of 10 mM sodium phosphate buffer at pH 6.5 and 4 μL of test compound solutions (5 mM in DMSO) or DMSO (for the reference wells) to give triplicate samples. The plate was shaken at room temperature for 1 hour then sat covered for 1 hour and the emission of the liposomes at 675 nm, 642 nm and 572 nm (excitation of 470 nm) was read. Chart 1 shows the emission of the liposomes exposed to compounds as well as the emission of the reference wells; the emission of liposomes exposed to compounds is significantly below that of the emission of reference liposomes (exposed only to buffer and DMSO) in all cases, despite the structural variation in the compound set. The test compounds include positive, neutral and negatively charged compounds with different functional groups and molecular weights.

[image]

These materials and method could be used in environmental monitoring as described in the following examples.

### EXAMPLE 5

The three or two-dimensional polydiacetylene arrays are exposed to air, which contains organic solvent vapors. The fluorescence of the arrays changes upon exposure to the air/vapor mixture, and will do so for a variety of vapors and upon exposure to vapor mixtures. The change in fluorescence signals the presence of the vapor(s) in air, but does not necessarily identify them.

### EXAMPLE 6

The three or two-dimensional polydiacetylene arrays are exposed to water samples, which contain chemicals (e.g. organic solvents, heavy metals, organometallic complexes, inorganic compounds, etc). The fluorescence of the arrays changes upon exposure to the water/chemicals, and will do so for a variety of solvents and upon exposure to water/chemicals mixture. The change in fluorescence signals the presence of chemicals in water, but does not necessarily identify them.

The disclosure shows and describes only the preferred embodiments but, as mentioned above, it is to be understood that it is capable of use in various other combinations, modifications, and environments and is capable of changes or modifications within the scope of the concept as expressed herein, commensurate with the above teachings and/or the skill or knowledge of the relevant art. The embodiments described hereinabove are further intended to explain best modes known to the present inventors and to enable others skilled in the art to utilize it in such, or other, embodiments and with the various modifications required by the particular applications or uses. Accordingly, the description is not intended to limit the invention to the form disclosed herein. Also, it is intended that the appended claims be construed to include alternative embodiments.

All publications and patent applications cited in this specification are herein incorporated by reference, and for any and all purposes, as if each individual publication or patent application were specifically and individually indicated to be incorporated by reference. In the case of inconsistencies the present disclosure will prevail.

What is claimed is:

1. A method for screening a plurality of samples containing different species, which comprises exposing a three-dimensional array of a polydiacetylene backbone or a two-dimensional array of a polydiacetylene backbone, or both, to the samples to be evaluated;
wherein the array is capable of hetero-detection;
detecting the change in fluorescence or phosphorescence of the array, and
comparing the change to a previously determined change in fluorescence or phosphorescence of the array.

2. The method of claim 1 wherein the screening comprises evaluating at least one of absorption, distribution, metabolism or excretion properties of the species in the samples.

3. The method of claim 1 wherein the screening comprises detecting permeability of a species in a sample across a membrane.

4. The method of claim 1 wherein the screening comprises measuring solubility.

5. The method of claim 1 wherein the screening comprises measuring compound bonding to a protein.

6. The method of claim 5 wherein the protein comprises human serum albumin.

7. The method of claim 1 wherein at least about five different samples are screened.

8. The method of claim 1 wherein at least about twenty different samples are screened.

9. The method of claim 1 wherein about at least one hundred different samples are screened.

10. The method of claim 1 wherein about at least one thousand different samples are screened.

11. The method of claim 1 wherein the array comprises a three-dimensional array in the form of a solution of liposomes, tubes, tubules, fibers or ribbons.

12. The method of claim 1 wherein the decrease in fluorescence of the polydiacetylene array is measured.

13. The method of claim 1 wherein the increase in fluorescence of the polydiacetylene array is measured.

14. The method of any one of claims 1, 11 12 or 13 wherein the three-dimensional array or a two-dimensional array further comprises a fluorophore and wherein the change in fluorescence of the polydiacetylene array is monitored.

15. The method of any one of claims 1, 11 12 or 13 wherein the three-dimensional array or a two-dimensional array further comprises a fluorophore and wherein the change in fluorescence of the fluorophore is monitored.

16. The method of claim 1 wherein array does not contain a further fluorophore.

17. The method of claim 1 wherein the change in fluorescence is detected by exposure to light having wavelengths below 550 nm and measurement of the emission.

18. The method of claim 1 wherein the change in fluorescence is detected by exposure to light having wavelengths between 450 and 500 nm and measurement of the emission.

19. The method of claim 1 wherein the array is located onto a solid support.

20. The method of claim 19 wherein the solid support is a porous membrane.

21. The method of claim 1 wherein the array is unsupported.

22. The method of claim 1 which comprises passing the sample through a permeable membrane and measuring the quantity of the material that passed through the membrane.

23. The method of claim 1 wherein the array is located on a non-porous support.
